(19) United States
(12) Patent Application Publication
(10) Pub. No.: US 2012/0301885 A1
(43) Pub. Date: Nov. 29, 2012

(54) CYTOGENIC ANALYSIS OF METAPHASE CHROMOSOMES

(76) Inventors: Lei Tang, Oro Valley, AZ (US); Hong Ni, Austin, TX (US); Heather Noelle Lewis, Oro Valley, AZ (US)

(21) Appl. No.: 13/576,142

(22) PCT Filed: Feb. 24, 2011
(86) PCT No.: PCT/US11/26027

§ 371 Date: Jul. 30, 2012

Related U.S. Application Data
(60) Provisional application No. 61/308,675, filed on Feb. 26, 2010.

(51) Int. Cl. G01N 21/78; C12Q 1/68

(52) U.S. Cl. 435/6.11

(57) ABSTRACT

The present invention relates to methods and systems for analyzing chromosomes, and in particular to methods and systems for simultaneously performing banding and in situ hybridization on metaphase chromosomes.

### CROSS-REFERENCE TO RELATED APPLICATIONS

The present invention claims priority to pending U.S. Provisional Patent Application No. 61/308,675, filed Feb. 26, 2010, the contents of which are hereby incorporated by reference in its entirety.

### FIELD OF THE INVENTION

The present invention relates to methods and systems for analyzing chromosomes, and in particular to methods and systems for simultaneously performing banding and in situ hybridization on metaphase chromosomes.

### BACKGROUND OF THE INVENTION

Standard cytogenetic studies allow a cytogeneticist to survey the whole genome for abnormalities of chromosome number or structure. The karyotype is the characteristic chromosome complement of a eukaryote species. Karyotypes are commonly used for several purposes, including the study of chromosomal aberrations, cellular function and taxonomic relationships.

Karyotyping typically involves the banding of chromosomes. There are several techniques for chromosome banding. G-banding is obtained with Giemsa stain following treatment of chromosomes with trypsin. G-banding results in chromosomes that are stained with alternating light and dark bands. The light regions tend to be euchromatic, early-replicating, and GC rich. The dark regions tend to be heterochromatic, late-replicating, and AT rich. R-banding (reverse banding) is the reverse of G-banding. The dark regions are euchromatic (GC rich) and the bright regions are heterochromatic (AT rich). Another type of banding is termed replication banding or fluorescence plus Giemsa (FPG) banding. Still other types of banding include C-banding, Q-banding and fluorescence banding.

Molecular cytogenetic techniques have also been developed. Molecular cytogenetic techniques have enabled more accurate and refined cytogenetic diagnoses, both for constitutional abnormalities and acquired changes in cancer cells. The most commonly used molecular cytogenetic techniques are various in situ hybridization (ISH) techniques, such as fluorescence in situ hybridization (FISH) and colorimetric in situ hybridization (CISH). In conventional ISH techniques, a nucleic acid probe labeled with a detectable label is hybridized to a denatured mitotic chromosome, thereby contacting a target nucleic acid sequence. The target nucleic acid sequence is then detected by detecting the label.

Several references have disclosed the combination of banding techniques with ISH. See, e.g., Garson et al., Novel non-isotopic in situ hybridization technique detects small (1 kb) unique sequences in routinely G-banded human chromosomes: fine mapping of N-myc and B-NGF genes, Nucl. Acids. Res. 15(12) 4761-70 (1987); Lemieux et al., A simple method for simultaneous R- or G-banding and fluorescence in situ hybridization of small single-copy genes, Cytogenet. Cell. Genetic 59(4):311-12 (1992); Shi et al., The mapping of transgenes by fluorescence in situ hybridization on G-banded mouse chromosomes, Mamml. Genome 5:337-41 (1994); Boyle et al. Rapid physical mapping of cloned DNA on banded mouse chromosomes by fluorescence in situ hybridization, Genomics 12 106-15 (1992); Larremendy et al., Simultaneous detection of high resolution R-banding and fluorescence in situ hybridization signals after fluorouracil induced cellular synchronization, Hereditas 119:89-94 (1994); Schook, Gene Mapping Techniques and Applications, 1991, Ch. 6, pg. 121-123; Bhatt et al., Nucleic Acids Research, 1988, Vol. 16, No. 9 3951-3961; Zhang et al., Chromosoma. 1990 October; 99(6):436-9; and Smit et al., Cytogenet Cell Genet 54:20-23 (1990).

However, the techniques described in these references are not efficient. The chromosome banding is performed before the ISH and the stain is washed off. The sample is imaged, and then ISH is performed. The sample must then be reimaged and aligned. The destaining and multiple imaging limit the utility of analyzing both chromosome structure and molecular characteristics of chromosomes in the same sample.

What is needed in the art are improved methods and systems for performing both a structural analysis of chromosomes and a molecular analysis of chromosomes in the same sample.

### SUMMARY OF THE INVENTION

The present invention relates to methods and systems for analyzing chromosomes, and in particular to methods and systems for simultaneously performing banding and in situ hybridization on metaphase chromosomes. In some embodiments, the present invention provides methods for in situ analysis of a sample comprising chromosomes, the method comprising: contacting the sample comprising chromosomes with at least one first probe specific for a first target nucleic acid in the chromosomes under conditions such that the probe hybridizes to the target nucleic acid, contacting the sample with in situ hybridization assay reagents, banding the chromosome to provide a banded chromosome, and simultaneously analyzing the banded chromosome for banding and hybridization of the probe specific for the target nucleic acid, wherein the presence of the probe on the chromosome is indicated by the in situ hybridization assay reagents. In some embodiments, the banding is performed by Giemsa staining the chromosome.

In some embodiments, the first probe specific for the first target nucleic acid is conjugated to an enzyme that reacts with a colorimetric substrate and the in situ hybridization assay reagents comprise the colorimetric substrate. In some embodiments, the first probe specific for the first target nucleic acid is conjugated with to a fluorescent moiety. In some embodiments, the enzyme that reacts with a colorimetric substrate is selected from the group consisting of horseradish peroxidase, alkaline phosphatase, acid phosphatase, glucose oxidase, β-galactosidase, β-glucuronidase and β-lactamase. In some embodiments, the colorimetric substrate is selected from the group consisting of diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

In some embodiments, the first probe specific for the first target nucleic acid is conjugated to a hapten, and the in situ hybridization assay reagents comprise a specific binding reagent that binds to the hapten, the specific binding reagent comprising a signal generating moiety. In some embodiments, the hapten is selected from the group consisting of biotin, 2,4-Dintropheyl (DNP), Fluorescein deratives, Digoxygenin (DIG), 5-Nitro-3-pyrozolecarbamide (nitropyrazole, NP), 4,5,-Dimethoxy-2-nitrocinnamide (nitrocinnamide, NCA), 2-(3,4-Dimethoxyphenyl)-quinoline-4-carbamide (phenylquinolone, DPQ), 2,1,3-Benzoxadiazole-5-carbamide (benzofurazan, BF), 3-Hydroxy-2-quinoxalinecarbamide (hydroxyquinoxaline, HQ), 4-(Dimethylamino)azobenzene-4′-sulfonamide (DABSYL), Rotenone isoxazoline (Rot), (E)-2-(2-(2-oxo-2,3-dihydro-1H-benzo[b][1,4]diazepin-4-yl)phenozy)acetamide (benzodiazepine, BD), 7-(diethylamino)-2-oxo-2H-chromene-3-carboxylic acid (coumarin 343, CDO), 2-Acetamido-4-methyl-5-thiazolesulfonamide (thiazolesulfonamide, TS), and p-Mehtoxyphenylpyrazopodophyllamide (Podo). In some embodiments, the specific binding agent is conjugated to a signal generating moiety comprising an enzyme selected from the group consisting of horseradish peroxidase, alkaline phosphatase, acid phosphatase, glucose oxidase, β-galactosidase, β-glucuronidase and β-lactamase.

In some embodiments, the sample comprising chromosomes is immobilized prior to the hybridization. In some embodiments, the chromosomes are immobilized by cross-linking comprising exposure to ultraviolet radiation. In some embodiments, the chromosomes are immobilized by cross-linking comprising exposure to a chemical cross-linking agent. In some embodiments, the chemical cross-linking agents are selected from the group consisting of formaldehyde, glutaraldehyde, dimethyl suberimidate, dimethyl adipimidate, and N-hydroxysuccinimide esters. In some embodiments, the sample comprising chromosomes is enzymatically treated prior to the hybridization step. In some embodiments, the enzymatic treatment comprises treatment with trypsin. In some embodiments, the analyzing comprises viewing the sample with a light microscope. In some embodiments, the analyzing comprises computer imaging the sample with a light microscope. In some embodiments, the sample comprises cells fixed on a substrate. In some embodiments, the cells are cells in a tissue section. In some embodiments, the methods further comprise contacting the sample comprising chromosomes with at least one second probe specific for a second target nucleic acid in the chromosomes under conditions such that the probe hybridizes to the target nucleic acid and detecting the second probe.

In some embodiments, the present invention provides methods for in situ analysis of a sample comprising chromosomes, the method comprising: cross-linking the sample comprising chromosomes; treating the sample comprising chromosomes with trypsin; contacting the sample comprising chromosomes with a probe specific for a target nucleic acid in the chromosomes under conditions such that the probe hybridizes to the target nucleic acid, contacting the sample with colorimetric assay reagents, banding the chromosome to provide a banded chromosome, and simultaneously analyzing the banded chromosome for banding and hybridization of the probe specific for the target nucleic acid, wherein the presence of the probe on the chromosome is indicated by the colorimetric assay reagents.

In some embodiments, the present invention provides automated systems for in situ analysis of a sample comprising chromosomes, the system comprising: substrates compatible with fixation of a sample comprising chromosomes; one or more probes specific for one or more target nucleic acids in the chromosomes; colorimetric assay reagents for detection of the probes; and banding reagents for banding the chromosomes.

In some embodiments, the present invention provides kits for in situ analysis of a sample comprising chromosomes, the system comprising: one or more probes specific for one or more target nucleic acids in the chromosomes; colorimetric assay reagents for detection of the probes; and banding reagents for banding the chromosomes.

### DESCRIPTION OF THE FIGURES

FIG. 1a and FIG. 1b are light micrographs of sample that hat has been ISH-stained and banded.

### DEFINITIONS

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” is used synonymously with the phrase “more than one,” that is, two or more. It is further to be understood that all base sizes or amino acid sizes, and all molecular weight or molecular mass values, given for nucleic acids or polypeptides are approximate, and are provided for description. The term “comprises” means “includes.” The abbreviation, “e.g.,” is derived from the Latin exempli gratia, and is used herein to indicate a non-limiting example. Thus, the abbreviation “e.g.,” is synonymous with the term “for example.” Although methods and materials similar or equivalent to those described herein can be used in the practice or testing of this disclosure, suitable methods and materials are described below.

In order to facilitate review of the various embodiments of this disclosure, the following explanations of specific terms are provided:

A nucleic acid molecule is said to be “complementary” with another nucleic acid molecule if the two molecules share a sufficient number of complementary nucleotides to form a stable duplex or triplex when the strands bind (hybridize) to each other, for example by forming Watson-Crick, Hoogsteen or reverse Hoogsteen base pairs. Stable binding occurs when a nucleic acid molecule remains detectably bound to a target nucleic acid sequence (e.g., genomic target nucleic acid sequence) under the required conditions.

Complementarity is the degree to which bases in one nucleic acid molecule (e.g., target nucleic acid probe) base pair with the bases in a second nucleic acid molecule (e.g., genomic target nucleic acid sequence). Complementarity is conveniently described by percentage, that is, the proportion of nucleotides that form base pairs between two molecules or within a specific region or domain of two molecules.

In the present disclosure, “sufficient complementarity” means that a sufficient number of base pairs exist between one nucleic acid molecule or region thereof and a target nucleic acid sequence (e.g., genomic target nucleic acid sequence) to achieve detectable binding. A thorough treatment of the qualitative and quantitative considerations involved in establishing binding conditions is provided by Beltz et al. Methods Enzymol. 100:266-285, 1983, and by Sambrook et al. (ed.), Molecular Cloning. A Laboratory Manual, 2nd ed., vol. 1-3, Cold Spring Harbor Laboratory Press, Cold Spring Harbor, N.Y., 1989.

The terms “conjugating, joining, bonding or linking” refer to covalently linking one molecule to another molecule to make a larger molecule. For example, making two polypeptides into one contiguous polypeptide molecule, or to covalently attaching a hapten or other molecule to a polypeptide, such as an scFv antibody. In the specific context, the terms include reference to joining a specific binding molecule such as an antibody to a signal generating moiety, such as a quantum dot. The linkage can be either by chemical or recombinant means. “Chemical means” refers to a reaction between the antibody moiety and the effector molecule such that there is a covalent bond formed between the two molecules to form one molecule.

The term “coupled”, when applied to a first atom or molecule being “coupled” to a second atom or molecule can be both directly coupled and indirectly coupled. A secondary antibody provides an example of indirect coupling. One specific example of indirect coupling is a rabbit anti-hapten primary antibody that is bound by a mouse anti-rabbit IgG antibody, that is in turn bound by a goat anti-mouse IgG antibody that is covalently linked to a detectable label.

The term “corresponding” in reference to a first and second nucleic acid (for example, a binding region and a target nucleic acid sequence) indicates that the first and second nucleic acid share substantial sequence identity or complementarity over at least a portion of the total sequence of the first and/or second nucleic acid. Thus, a binding region corresponds to a target nucleic acid sequence if the binding region possesses substantial sequence identity or complementarity (e.g., reverse complementarity) with (e.g., if it is at least 80%, at least 85%, at least 90%, at least 95%, or even 100% identical or complementary to) at least a portion of the target nucleic acid sequence. For example, a binding region can correspond to a target nucleic acid sequence if the binding region possesses substantial sequence identity to one strand of a double-stranded target nucleic acid sequence (e.g., genomic target DNA sequence) or if the binding region is substantially complementary to a single-stranded target nucleic acid sequence (e.g. RNA or an RNA viral genome).

A “genome” is the total genetic constituents of an organism. In the case of eukaryotic organisms, the genome is contained in a haploid set of chromosomes of a cell. In the case of prokaryotic organisms, the genome is contained in a single chromosome, and in some cases one or more extra-chromosomal genetic elements, such as episomes (e.g., plasmids). A viral genome can take the form of one or more single or double stranded DNA or RNA molecules depending on the particular virus.

The term “hapten” refers to a molecule, typically a small molecule that can combine specifically with an antibody, but typically is substantially incapable of being immunogenic except in combination with a carrier molecule.

The term “isolated” in reference to a biological component (such as a nucleic acid molecule, protein, or cell), refers to a biological component that has been substantially separated or purified away from other biological components in the cell of the organism, or the organism itself, in which the component naturally occurs, such as other chromosomal and extra-chromosomal DNA and RNA, proteins, cells, and organelles. Nucleic acid molecules that have been “isolated” include nucleic acid molecules purified by standard purification methods. The term also encompasses nucleic acids prepared by amplification or cloning as well as chemically synthesized nucleic acids.

A “label” is a detectable compound or composition that is conjugated directly or indirectly to another molecule to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable). Exemplary labels in the context of the probes disclosed herein are described below. Methods for labeling nucleic acids, and guidance in the choice of labels useful for various purposes, are discussed, e.g., in Sambrook and Russel, in Molecular Cloning: A Laboratory Manual, 3rd Ed., Cold Spring Harbor Laboratory Press (2001) and Ausubel et al., in Current Protocols in Molecular Biology, Greene Publishing Associates and Wiley-Intersciences (1987, and including updates).

The term “multiplex” refers to embodiments that allow multiple targets in a sample to be detected substantially simultaneously, or sequentially, as desired, using plural different conjugates. Multiplexing can include identifying and/or quantifying nucleic acids generally, DNA, RNA, peptides, proteins, both individually and in any and all combinations. Multiplexing also can include detecting two or more of a gene, a messenger and a protein in a cell in its anatomic context.

A “nucleic acid” is a deoxyribonucleotide or ribonucleotide polymer in either single or double stranded form, and unless otherwise limited, encompasses analogues of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in a polynucleotide. A nucleotide sequence refers to the sequence of bases in a polynucleotide.

A “probe” or a “nucleic acid probe” is a nucleic acid molecule that is capable of hybridizing with a target nucleic acid molecule (e.g., genomic target nucleic acid molecule) and, when hybridized to the target, is capable of being detected either directly or indirectly. Thus probes permit the detection, and in some examples quantification, of a target nucleic acid molecule. In particular examples a probe includes a plurality of nucleic acid molecules, which include binding regions derived from the target nucleic acid molecule and are thus capable of specifically hybridizing to at least a portion of the target nucleic acid molecule. A probe can be referred to as a “labeled nucleic acid probe,” indicating that the probe is coupled directly or indirectly to a detectable moiety or “label,” which renders the probe detectable.

The term “quantum dot” refers to a nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. A variety of quantum dots having various surface chemistries and fluorescence characteristics are commercially available from Invitrogen Corporation, Eugene, Oreg. (see, for example, U.S. Pat. Nos. 6,815,064, 6,682,596 and 6,649,138, each of which patents is incorporated by reference herein). Quantum dots are also commercially available from Evident Technologies (Troy, N.Y.). Other quantum dots include alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN quantum dots (Alloy quantum dots and methods for making the same are disclosed, for example, in US Application Publication No. 2005/0012182 and PCT Publication WO 2005/001889).

A “sample” is a biological specimen containing genomic DNA, RNA (including mRNA), protein, or combinations thereof, obtained from a subject. Examples include, but are not limited to, chromosomal preparations, peripheral blood, urine, saliva, tissue biopsy, surgical specimen, bone marrow, amniocentesis samples and autopsy material. In one example, a sample includes genomic DNA or RNA. In some examples, the sample is a cytogenetic preparation, for example which can be placed on microscope slides. In particular examples, samples are used directly, or can be manipulated prior to use, for example, by fixing (e.g., using formalin).

The term “signal generating moiety” refers to a composition or molecule that geberates a signal that is detectable by an assay.

The term “specific binding moiety” refers to a member of a binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A), nucleic acids sequences, and protein-nucleic acids. Specific binding moieties can also include the molecules (or portions thereof) that are specifically bound by such specific binding proteins.

The term “specific binding agent” refers to a molecule that comprises a specific binding moiety conjugated to a signal generating moiety.

A “subject” includes any multi-cellular vertebrate organism, such as human and non-human mammals (e.g., veterinary subjects).

A “target nucleic acid sequence or molecule” is a defined region or particular sequence of a nucleic acid molecule, for example a genome (such as a gene or a region of mammalian genomic DNA containing a gene of interest) or an RNA sequence. In an example where the target nucleic acid sequence is a target genomic sequence, such a target can be defined by its position on a chromosome (e.g., in a normal cell), for example, according to cytogenetic nomenclature by reference to a particular location on a chromosome; by reference to its location on a genetic map; by reference to a hypothetical or assembled contig; by its specific sequence or function; by its gene or protein name, or by any other means that uniquely identifies it from among other genetic sequences of a genome. In some examples, the target nucleic acid sequence is mammalian or viral genomic sequence. In other examples, the target nucleic acid sequence is an RNA sequence.

In some examples, alterations of a target nucleic acid sequence (e.g., genomic nucleic acid sequence) are “associated with” a disease or condition. That is, detection of the target nucleic acid sequence can be used to infer the status of a sample with respect to the disease or condition. For example, the target nucleic acid sequence can exist in two (or more) distinguishable forms, such that a first form correlates with absence of a disease or condition and a second (or different) form correlates with the presence of the disease or condition. The two different forms can be qualitatively distinguishable, such as by polynucleotide polymorphisms, and/or the two different forms can be quantitatively distinguishable, such as by the number of copies of the target nucleic acid sequence that are present in a cell.

### DETAILED DESCRIPTION OF THE INVENTION

The present invention relates to methods and systems for analyzing chromosomes, and in particular to methods and systems for simultaneously performing banding and in situ hybridization on metaphase chromosomes. The present invention provides methods and systems for chromosome banding and ISH so that the results of both the chromosome banding and ISH can be analyzed simultaneously, for example by microscopy. These methods and systems allow for faster, more convenient, and more accurate diagnosis of chromosome abnormalities. The systems and methods can also be used to test for nucleic acid probe sensitivity and specificity as well as for quality control during probe production.

### A. In Situ Hybridization and Chromosome Banding

The present invention provides systems and methods for ISH and banding of chromosomes preparations. The techniques of the present invention may be used with a wide variety of samples. For example, the samples may be cells or tissues from any eukaryotic organism. In some preferred embodiments, the cells are tissues are from a human or from an animal of research, veterinary or commercial interest such as mouse, rat, dog, cat, bird, horse, goat, cow or sheep. In some embodiments, the samples are mounted on a solid substrate such as a microscope slide. In some embodiments, the samples are cross sections of tissues. In other embodiments, the samples are cells that have been obtained from the organism. For example, the sample can be cross sections fixed in paraffin, formalin-fixed tissue, blood or bone marrow smears, and directly fixed cells or other nuclear isolates. In some embodiments, the sample is from a subject that is suspected of having a disease or disorder. For example, the sample may come from a subject suspected of having a constitutive genetic anomaly, such as a microdeletion syndrome, a chromosome translocation, gene amplification or aneuploidy syndromes, a neoplastic disease, or a pathogen infection. In some embodiments, the techniques herein are used to characterize tumor cells for both diagnosis and prognosis of cancer. Numerous chromosomal abnormalities have been associated with the development of cancer (for example, aneuploidies such as trisomy 8 associated with certain myeloid disorders; translocations such as the BCR/ABL rearrangement in chronic myelogenous leukemia; and amplifications of specific nucleic acid sequences associated with neoplastic transformation). The techniques of the present invention are useful for analyzing these chromosomal abnormalities. Accordingly, in some embodiments, the samples are from a patient that is suspected of having cancer or has been diagnosed with cancer. In some embodiments, the samples are tissue or cell biopsies from a subject suspected of having cancer or that has cancer.

The samples are preferably treated prior to the ISH and chromosome banding procedures. In some embodiments, the samples, preferably provided on a substrate such as a microscope slide, are cross-linked. The samples may be cross-linked by any suitable procedure. Examples of cross-linking procedures include, but are not limited to, ultraviolet (UV) cross-linking in which the sample is exposed to UV radiation and chemical cross-linking. In some embodiments, the UV cross-linking procedure comprises exposing the sample to UV radiation for a predetermined period of time and predetermined energy. For example, the sample may be exposed to UV radiation for a period of time from about 10 seconds to about 10 minutes, at an energy of from about 50 to about 500 mJ, preferably about 150 to 250 mJ, and most preferably at about 200 mJ. Commercial systems are available for UV cross-linking. In some embodiments, a Stratalinker 2400 (Stratagene Model # C00518) is utilized for UV cross-linking Examples of suitable chemical cross-linking procedures include, but are not limited to, treatment with chemical cross-linking agents such as formaldehyde, glutaraldehyde, dimethyl suberimidate, dimethyl adipimidate, N-hydroxysuccinimide esters, and the like, including both homobifunctional and heterobifunctional cross-linkers.

The samples are also preferably treated with enzymes prior to the ISH and chromosome banding procedures. In some embodiments, the enzymatic treatment comprises treatment with protease. Suitable proteases include trypsin, chymotrypsin, calpain, capsase, cathepsin, papain and the like. In some embodiments, the samples are enzymatically treated for a predetermined time and with a predetermined concentration or enzyme. In some embodiments, for example, the sample is treated with a solution comprising trypsin in a concentration for from about 0.05% to about 2% for from about 1 to about 20 minutes.

ISH and chromosome banding are then performed on the treated samples. In some embodiments, the ISH is performed using an automated instrument. Ventana Medical Systems, Inc. is the assignee of a number of U.S. patents disclosing systems and methods for performing automated analyses, including U.S. Pat. Nos. 5,650,327, 5,654,200, 6,296,809, 6,352,861, 6,827,901 and 6,943,029, and U.S. published application Nos. 20030211630 and 20040052685, each of which is incorporated herein by reference. Particular embodiments of ISH procedures can be conducted using various automated processes. Additional details concerning exemplary working embodiments are provided in the working examples and in the product literature. In some embodiments, the automated ISH system is a Ventana BenchMark XT™ instrument. The present invention is not limited to the use of any particular ISH procedure or type of labeled probe. Suitable ISH procedures include, but are not limited to, fluorescence in situ hybridization (FISH), chromogenic in situ hybridization (CISH) and silver in situ hybridization (SISH)).

In general, hybridization between complementary nucleic acid molecules is mediated via hydrogen bonding, which includes Watson-Crick, Hoogsteen or reversed Hoogsteen hydrogen bonding between complementary nucleotide units. For example, adenine and thymine are complementary nucleobases that pair through formation of hydrogen bonds. If a nucleotide unit at a certain position of a probe of the present disclosure is capable of hydrogen bonding with a nucleotide unit at the same position of a DNA or RNA molecule (e.g., a target nucleic acid sequence) then the oligonucleotides are complementary to each other at that position. The probe and the DNA or RNA are complementary to each other when a sufficient number of corresponding positions in each molecule are occupied by nucleotide units which can hydrogen bond with each other, and thus produce detectable binding. A probe need not be 100% complementary to its target nucleic acid sequence (e.g., genomic target nucleic acid sequence) to be specifically hybridizable. However sufficient complementarity is needed so that the probe binds, duplexes, or hybridizes only or substantially only to a target nucleic acid sequence when that sequence is present in a complex mixture (e.g., total cellular DNA or RNA).

In situ hybridization involves contacting a sample containing a target nucleic acid sequence (e.g., genomic target nucleic acid sequence) in the context of a metaphase or interphase chromosome preparation (such as a cell or tissue sample mounted on a slide) with a probe (i.e., a target nucleic acid probe) specifically hybridizable or specific for the target nucleic acid sequence (e.g., genomic target nucleic acid sequence). The slides are optionally pretreated, e.g., to remove paraffin or other materials that can interfere with uniform hybridization. The chromosome sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess target nucleic acid probe, and detection of specific labeling of the chromosome target is performed. For a general description of in situ hybridization procedures, see, e.g., U.S. Pat. No. 4,888,278. Numerous procedures for fluorescence in situ hybridization (FISH), chromogenic in situ hybridization (CISH) and silver in situ hybridization (SISH) are known in the art. For example, procedures for performing FISH are described in U.S. Pat. Nos. 5,447,841, 5,472,842, 5,427,932, and for example, in Pinkel et al., Proc. Natl. Acad. Sci. 83:2934-2938, 1986; Pinkel et al., Proc. Natl. Acad. Sci. 85:9138-9142, 1988, and Lichter et al., Proc. Natl. Acad. Sci. 85:9664-9668, 1988. CISH is described in, e.g., Tanner et al., Am. J. Pathol. 157:1467-1472, 2000, and U.S. Pat. No. 6,942,970. Additional detection methods are provided in U.S. Pat. No. 6,280,929. Exemplary procedures for detecting viruses by in situ hybridization can be found in Poddighe et al., J. Clin. Pathol. 49:M340-M344, 1996.

Numerous reagents and detection schemes can be employed in conjunction with FISH, CISH, and SISH procedures to improve sensitivity, resolution, or other desirable properties. For example, target nucleic acid probes comprising a signal-generating such as an enzyme, fluorochrome, or quantum dot can be optically detected. In some embodiments, target nucleic acid probe can be labeled with a detectable moiety, such as a hapten (such as the following non-limiting examples: biotin, digoxygenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof, and in particular, 2,4-Dintropheyl (DNP), Biotin, Fluorescein deratives (FITC, TAMRA, Texas Red, etc.), Digoxygenin (DIG), 5-Nitro-3-pyrozolecarbamide (nitropyrazole, NP), 4,5,-Dimethoxy-2-nitrocinnamide (nitrocinnamide, NCA), 2-(3,4-Dimethoxyphenyl)-quinoline-4-carbamide (phenylquinolone, DPQ), 2,1,3-Benzoxadiazole-5-carbamide (benzofurazan, BF), 3-Hydroxy-2-quinoxalinecarbamide (hydroxyquinoxaline, HQ), 4-(Dimethylamino)azobenzene-4′-sulfonamide (DABSYL), Rotenone isoxazoline (Rot), (E)-2-(2-(2-oxo-2,3-dihydro-1H-benzo[b][1,4]diazepin-4-yl)phenozy)acetamide (benzodiazepine, BD), 7-(diethylamino)-2-oxo-2H-chromene-3-carboxylic acid (coumarin 343, CDO), 2-Acetamido-4-methyl-5-thiazolesulfonamide (thiazolesulfonamide, TS), and p-Mehtoxyphenylpyrazopodophyllamide (Podo)), ligand or other indirectly detectable moiety. Target nucleic acid probes labeled with such molecules (and the target nucleic acid sequences to which they bind) can then be detected by contacting the sample (e.g., the cell or tissue sample to which the probe is bound) with a labeled specific binding reagent, such as an antibody (or receptor, or other specific binding partner) specific for the chosen detectable moiety.

It will be appreciated by those of skill in the art that by appropriately selecting labeled detection probes and/or labeled detectable moiety/specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first detection probe that corresponds to a first target nucleic acid probe can be labeled with a first hapten, such as biotin, while a second detection probe that corresponds to a second target nucleic acid sequence can be labeled with a second hapten, such as DNP. Following exposure of the sample to the probe sets, the bound probes can be detected by contacting the sample with a first specific binding agent (in this case avidin labeled with a first enzyme) and a second specific binding agent (in this case an anti-DNP antibody, or antibody fragment, labeled with a second enzyme). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

In some embodiments, the binding agent that is specific for a target nucleic acid probe (such as an antibody, e.g., a primary antibody, receptor or other binding agent) is conjugated to an enzyme that is capable of converting a fluorogenic or chromogenic composition into a detectable fluorescent, colored or otherwise detectable signal (e.g., development of a detectable chromogen is CISH). The enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and U.S. Provisional Patent Application No. 60/739,794. Suitable enzymes that can serve as signal generating moieties include, but are not limited to, horseradish peroxidase, alkaline phosphatase, acid phosphatase, glucose oxidase, β-galactosidase, β-glucuronidase or β-lactamase. Where the detectable label includes an enzyme, a chromogen, fluorogenic compound, or luminogenic compound can be used in combination with the enzyme to generate a detectable signal (numerous of such compounds are commercially available, for example, from Invitrogen Corporation, Eugene Oreg.). Particular examples of chromogenic compounds include, but are not limited to, diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

In some embodiments, the target nucleic acid probe or its specific binding agent are labeled by a fluorophore for use in FISH. Examples of particular fluorophores that can be attached (for example, chemically conjugated) to a nucleic acid molecule or protein such as an antigen binding molecule include, but are not limited to, 4-acetamido-4′-isothiocyanatostilbene-2,2′ disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); 2′,7′-difluorofluorescein (OREGON GREEN™); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Invitrogen Detection Technologies, Molecular Probes (Eugene, Oreg.) and including the ALEXA FLUOR™ series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from QuantumDot Corp, Invitrogen Nanocrystal Technologies, Eugene, Oreg.; see also, U.S. Pat. Nos. 6,815,064, 6,682,596 and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et. al. (1998) Science 281:2013-6, Chan et al. (1998) Science 281:2016-8, and U.S. Pat. No. 6,274,323.

Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Invitrogen.

The samples are subjected to a chromosome banding process. In some embodiments, the chromosomes banded after the ISH process. In some embodiments, the sample is stained with Giemsa stain. In some embodiments, sample is contacted with a solution containing from about 0.5% to about 10% Giemsa, preferably about 4.0% Giemsa in an appropriate buffer. Appropriate buffers include, for example, Gurr buffer (Gibco, cat#10582-013). The sample is incubated in the solution for a period of time sufficient to stain the chromosomes in the sample. Suitable conditions, for example, comprise incubation at from about 20 C to about 50 C for from about 1 to about 10 minutes. Following staining, the slides are preferably rinsed and are ready for analysis, for example, by a microscope.

Standard light microscopes are an inexpensive tool for the detection of reagents and probes utilized in the CISH methods described above (fluorescent microscopes are used with FISH protocols). In some preferred embodiments, the microscopes are equipped with a computer imaging system for capturing and storing images of sample following ISH and banding. Accordingly, the present invention provides simplified methods for ISH and chromosome banding wherein metaphase chromosomes are prepared in a way that allows a hybridization of target nucleic acid probes immediately followed by Giemsa staining. In some preferred embodiments, the sample is cross-linked and treated with protease (e.g., trypsin) prior to ISH, and the sample is stained with Giemsa after ISH. In preferred embodiments, both signals (probe signal & banding) can be detected at the same time using only one instrument, e.g., a light microscope.

### B. Samples

The samples upon which the procedures of the present invention are performed comprise a target nucleic acid molecule. A target nucleic acid molecule can be any selected nucleic acid, such as DNA or RNA. In particular embodiments, the target sequence is a genomic target sequence or genomic subsequence, for example from a eukaryotic genome, such as a human genome. In some embodiments, the target nucleic acid is cytoplasmic RNA. In some embodiments, the target nucleic acid molecule is selected from a pathogen, such as a virus, bacteria, or intracellular parasite, such as from a viral genome. In some embodiments, the target nucleic acid sequence is a genomic sequence, such as eukaryotic (e.g., mammalian) or viral genomic sequence. Target nucleic acid probes can be generated which correspond to essentially any genomic target sequence that includes at least a portion of unique non-repetitive DNA. For example, the genomic target sequence can be a portion of a eukaryotic genome, such as a mammalian (e.g., human), fungal or intracellular parasite genome. Alternatively, a genomic target sequence can be a viral or prokaryotic genome (such as a bacterial genome), or portion thereof. In a specific example, the genomic target sequence is associated with an infectious organism (e.g., virus, bacteria, fungi).

In some embodiments, the target nucleic acid molecule can be a sequence associated with (e.g., correlated with, causally implicated in, etc.) a disease. In some embodiments, a target sequence is selected that is associated with a disease or condition, such that detection of hybridization can be used to infer information (such as diagnostic or prognostic information for the subject from whom the sample is obtained) relating to the disease or condition. In certain embodiments, the selected target nucleic acid molecule is a target nucleic acid molecule associated with a neoplastic disease (or cancer). In some embodiments, the genomic target sequence can include at least one at least one gene associated with cancer (e.g., HER2, c-Myc, n-Myc, Ab1, Bc12, Bc16, R1, p53, EGFR, TOP2A, MET, or genes encoding other receptors and/or signaling molecules, etc.) or chromosomal region associated with a cancer. In some embodiments, the target nucleic acid sequence can be associated with a chromosomal structural abnormality, e.g., a translocation, deletion, or reduplication (e.g., gene amplification or polysomy) that has been correlated with a cancer. In some embodiments, the target nucleic acid sequence encompasses a genomic sequence that is reduplicated or deleted in at least some neoplastic cells. The target nucleic acid sequence can vary substantially in size, such as at least 20 base pairs in length, at least 100 base pairs in length, at least 1000 base pairs in length, at least 50,000, at least 100,000, or even at least 250,000 base pairs in overall length.

The target nucleic acid sequence (e.g., genomic target nucleic acid sequence) can span any number of base pairs. In some embodiments, the target nucleic acid sequence spans at least 1000 base pairs. In specific examples, a target nucleic acid sequence (e.g., genomic target nucleic acid sequence) is at least 10,000, at least 50,000, at least 100,000, at least 150,000, at least 250,000, or at least 500,000 base pairs in length (such as 100 kb to 600 kb, 200 kb to 500 kb, or 300 kb to 500 kb). In examples, where the target nucleic acid sequence is from a eukaryotic genome (such as a mammalian genome, e.g., a human genome), the target sequence typically represents a small portion of the genome (or a small portion of a single chromosome) of the organism (for example, less than 20%, less than 10%, less than 5%, less than 2%, or less than 1% of the genomic DNA (or a single chromosome) of the organism). In some examples where the target sequence (e.g., genomic target nucleic acid sequence) is from an infectious organism (such as a virus), the target sequence can represent a larger proportion (for example, 50% or more) or even all of the genome of the infectious organism.

In specific non-limiting examples, a target nucleic acid sequence (e.g., genomic target nucleic acid sequence) associated with a neoplasm (for example, a cancer) is selected. Numerous chromosome abnormalities (including translocations and other rearrangements, reduplication or deletion) have been identified in neoplastic cells, especially in cancer cells, such as B cell and T cell leukemias, lymphomas, breast cancer, colon cancer, neurological cancers and the like. Therefore, in some examples, at least a portion of the target nucleic acid sequence (e.g., genomic target nucleic acid sequence) is reduplicated or deleted in at least a subset of cells in a sample.

Translocations involving oncogenes are known for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes nucleic acid molecules generated from a target nucleic acid sequence that extends distally from the SYT gene, and the second probe includes nucleic acid generated from a target nucleic acid sequence that extends 3′ or proximal to the SYT gene. When probes corresponding to these target nucleic acid sequences (e.g., genomic target nucleic acid sequences) are used in an in situ hybridization procedure, normal cells, which lacks a t(18q11.2) in the SYT gene region, exhibit two fusion (generated by the two labels in close proximity) signals, reflecting the two intact copies of SYT. Abnormal cells with a t(18q11.2) exhibit a single fusion signal.

Numerous examples of reduplication of genes involved in neoplastic transformation have been observed, and can be detected cytogenetically by in situ hybridization. In one example, a target nucleic acid sequence (e.g., genomic target nucleic acid sequence) is selected that includes a gene (e.g., an oncogene) that is reduplicated in one or more malignancies (e.g., a human malignancy). For example, HER2, also known as c-erbB2 or HER2/neu, is a gene that plays a role in the regulation of cell growth (a representative human HER2 genomic sequence is provided at GENBANK™ Accession No. NC_{—}000017, nucleotides 35097919-35138441). The gene codes for a 185 kd transmembrane cell surface receptor that is a member of the tyrosine kinase family. HER2 is amplified in human breast, ovarian, and other cancers. Therefore, a HER2 gene (or a region of chromosome 17 that includes the HER2 gene) can be used as a genomic target nucleic acid sequence to generate probes that include nucleic acid molecules with binding regions specific for HER2.

In other examples, a target nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1)) are characteristic molecular features of certain types of solid tumors of the central nervous system.

The aforementioned examples are provided solely for purpose of illustration and are not intended to be limiting. Numerous other cytogenetic abnormalities that correlate with neoplastic transformation and/or growth are known to those of skill in the art. Target nucleic acid sequences (e.g., genomic target nucleic acid sequences), which have been correlated with neoplastic transformation and which are useful in the disclosed methods and for which disclosed probes can be prepared, also include the EGFR gene (7p12; e.g., GENBANK™ Accession No. NC_{—}000007, nucleotides 55054219-55242525), the C-MYC gene (8q24.21; e.g., GENBANK™ Accession No. NC_{—}000008, nucleotides 128817498-128822856), D5S271 (5p15.2), lipoprotein lipase (LPL) gene (8p22; e.g., GENBANK™ Accession No. NC_{—}000008, nucleotides 19841058-19869049), RB1 (13q14; e.g., GENBANK™ Accession No. NC_{—}000013, nucleotides 47775912-47954023), p53 (17p13.1; e.g., GENBANK™ Accession No. NC_{—}000017, complement, nucleotides 7512464-7531642)), N-MYC (2p24; e.g., GENBANK™ Accession No. NC_{—}000002, complement, nucleotides 151835231-151854620), CHOP (12q13; e.g., GENBANK™ Accession No. NC_{—}000012, complement, nucleotides 56196638-56200567), FUS (16p11.2; e.g., GENBANK™ Accession No. NC_{—}000016, nucleotides 31098954-31110601), FKHR (13p14; e.g., GENBANK™ Accession No. NC_{—}000013, complement, nucleotides 40027817-40138734), as well as, for example: ALK (2p23; e.g., GENBANK™ Accession No. NC_{—}000002, complement, nucleotides 29269144-29997936), Ig heavy chain, CCND1 (11q13; e.g., GENBANK™ Accession No. NC_{—}000011, nucleotides 69165054 . . . 69178423), BCL2 (18q21.3; e.g., GENBANK™ Accession No. NC_{—}000018, complement, nucleotides 58941559-59137593), BCL6 (3q27; e.g., GENBANK™ Accession No. NC_{—}000003, complement, nucleotides 188921859-188946169), MALF1, AP1 (1p32-p31; e.g., GENBANK™ Accession No. NC_{—}000001, complement, nucleotides 59019051-59022373), TOP2A (17q21-q22; e.g., GENBANK™ Accession No. NC_{—}000017, complement, nucleotides 35798321-35827695), TMPRSS (21q22.3; e.g., GENBANK™ Accession No. NC_{—}000021, complement, nucleotides 41758351-41801948), ERG (21q22.3; e.g., GENBANK™ Accession No. NC_{—}000021, complement, nucleotides 38675671-38955488); ETV1 (7p21.3; e.g., GENBANK™ Accession No. NC_{—}000007, complement, nucleotides 13897379-13995289), EWS (22q12.2; e.g., GENBANK™ Accession No. NC_{—}000022, nucleotides 27994271-28026505); FLI1(11q24.1-q24.3; e.g., GENBANK™ Accession No. NC_{—}000011, nucleotides 128069199-128187521), PAX3 (2q35-q37; e.g., GENBANK™ Accession No. NC_{—}000002, complement, nucleotides 222772851-222871944), PAX7 (1p36.2-p36.12; e.g., GENBANK™ Accession No. NC_{—}000001, nucleotides 18830087-18935219, PTEN (10q23.3; e.g., GENBANK™ Accession No. NC_{—}000010, nucleotides 89613175-89716382), AKT2 (19q13.1-q13.2; e.g., GENBANK™ Accession No. NC_{—}000019, complement, nucleotides 45431556-45483036), MYCL1 (1p34.2; e.g., GENBANK™ Accession No. NC_{—}000001, complement, nucleotides 40133685-40140274), REL (2p13-p12; e.g., GENBANK™ Accession No. NC_{—}000002, nucleotides 60962256-61003682) and CSF1R (5q33-q35; e.g., GENBANK™ Accession No. NC_{—}000005, complement, nucleotides 149413051-149473128). A disclosed target nucleic acid probe or method may include a region of the respective human chromosome containing at least any one (or more, as applicable) of the foregoing genes. For example, the target nucleic acid sequence for some disclosed probes or methods includes any one of the foregoing genes and sufficient additional contiguous genomic sequence (whether 5′ of the gene, 3′ of the gene, or a combination thereof) for a total of at least 100,000 base pairs (such as at least 250,000, or at least 500,000 base pairs) or a total of between 100,000 and 500,000 base pairs.

In certain embodiments, the probe specific for the target nucleic acid molecule is assayed (in the same or a different but analogous sample) in combination with a second probe that provides an indication of chromosome number, such as a chromosome specific (e.g., centromere) probe. For example, a probe specific for a region of chromosome 17 containing at least the HER2 gene (a HER2 probe) can be used in combination with a CEP 17 probe that hybridizes to the alpha satellite DNA located at the centromere of chromosome 17 (17p11.1-q11.1). Inclusion of the CEP 17 probe allows for the relative copy number of the HER2 gene to be determined. For example, normal samples will have a HER2/CEP17 ratio of less than 2, whereas samples in which the HER2 gene is reduplicated will have a HER2/CEP17 ratio of greater than 2.0. Similarly, CEP centromere probes corresponding to the location of any other selected genomic target sequence can also be used in combination with a probe for a unique target on the same (or a different) chromosome.

In other examples, a target nucleic acid sequence (e.g., genomic target nucleic acid sequence) is selected from a virus or other microorganism associated with a disease or condition. Detection of the virus- or microorganism-derived target nucleic acid sequence (e.g., genomic target nucleic acid sequence) in a cell or tissue sample is indicative of the presence of the organism. For example, the probe can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some examples, the target nucleic acid sequence (e.g., genomic target nucleic acid sequence) is a viral genome. Exemplary viruses and corresponding genomic sequences (GENBANK™ RefSeq Accession No. in parentheses) include human adenovirus A (NC_{—}001460), human adenovirus B (NC_{—}004001), human adenovirus C(NC_{—}001405), human adenovirus D (NC_{—}002067), human adenovirus E (NC_{—}003266), human adenovirus F (NC_{—}001454), human astrovirus (NC_{—}001943), human BK polyomavirus (V01109; GI:60851) human bocavirus (NC_{—}007455), human coronavirus 229E (NC_{—}002645), human coronavirus HKU1 (NC_{—}006577), human coronavirus NL63 (NC_{—}005831), human coronavirus OC43 (NC_{—}005147), human enterovirus A (NC_{—}001612), human enterovirus B (NC_{—}001472), human enterovirus C(NC_{—}001428), human enterovirus D (NC_{—}001430), human erythrovirus V9 (NC_{—}004295), human foamy virus (NC_{—}001736), human herpesvirus 1 (Herpes simplex virus type 1) (NC_{—}001806), human herpesvirus 2 (Herpes simplex virus type 2) (NC_{—}001798), human herpesvirus 3 (Varicella zoster virus) (NC_{—}001348), human herpesvirus 4 type 1 (Epstein-Barr virus type 1) (NC_{—}007605), human herpesvirus 4 type 2 (Epstein-Barr virus type 2) (NC_{—}009334), human herpesvirus 5 strain AD169 (NC_{—}001347), human herpesvirus 5 strain Merlin Strain (NC_{—}006273), human herpesvirus 6A (NC_{—}001664), human herpesvirus 6B (NC_{—}000898), human herpesvirus 7 (NC_{—}001716), human herpesvirus 8 type M (NC_{—}003409), human herpesvirus 8 type P (NC_{—}009333), human immunodeficiency virus 1 (NC_{—}001802), human immunodeficiency virus 2 (NC_{—}001722), human metapneumovirus (NC_{—}004148), human papillomavirus-1 (NC_{—}001356), human papillomavirus-18 (NC._{—}001357), human papillomavirus-2 (NC_{—}001352), human papillomavirus-54 (NC_{—}001676), human papillomavirus-61 (NC_{—}001694), human papillomavirus-cand90 (NC_{—}004104), human papillomavirus RTRX7 (NC_{—}004761), human papillomavirus type 10 (NC_{—}001576), human papillomavirus type 101 (NC_{—}008189), human papillomavirus type 103 (NC_{—}008188), human papillomavirus type 107 (NC_{—}009239), human papillomavirus type 16 (NC_{—}001526), human papillomavirus type 24 (NC_{—}001683), human papillomavirus type 26 (NC_{—}001583), human papillomavirus type 32 (NC_{—}001586), human papillomavirus type 34 (NC_{—}001587), human papillomavirus type 4 (NC_{—}001457), human papillomavirus type 41 (NC_{—}001354), human papillomavirus type 48 (NC_{—}001690), human papillomavirus type 49 (NC_{—}001591), human papillomavirus type 5 (NC_{—}001531), human papillomavirus type 50 (NC_{—}001691), human papillomavirus type 53 (NC_{—}001593), human papillomavirus type 60 (NC_{—}001693), human papillomavirus type 63 (NC_{—}001458), human papillomavirus type 6b (NC_{—}001355), human papillomavirus type 7 (NC_{—}001595), human papillomavirus type 71 (NC_{—}002644), human papillomavirus type 9 (NC_{—}001596), human papillomavirus type 92 (NC_{—}004500), human papillomavirus type 96 (NC_{—}005134), human parainfluenza virus 1 (NC_{—}003461), human parainfluenza virus 2 (NC_{—}003443), human parainfluenza virus 3 (NC_{—}001796), human parechovirus (NC_{—}001897), human parvovirus 4 (NC_{—}007018), human parvovirus B19 (NC_{—}000883), human respiratory syncytial virus (NC_{—}001781), human rhinovirus A (NC_{—}001617), human rhinovirus B (NC_{—}001490), human spumaretrovirus (NC_{—}001795), human T-lymphotropic virus 1 (NC_{—}001436), human T-lymphotropic virus 2 (NC_{—}001488).

In certain examples, the target nucleic acid sequence (e.g., genomic target nucleic acid sequence) is from an oncogenic virus, such as Epstein-Barr Virus (EBV) or a Human Papilloma Virus (HPV, e.g., HPV16, HPV18). In other examples, the target nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., SARS virus), an Adenovirus, a Polyomavirus, a Cytomegalovirus (CMV), or a Herpes Simplex Virus (HSV).

### C. Kits and Systems

In some embodiments, the present invention provides kits for ISH and chromosome banding including at least one target nucleic acid probe, reagents for ISH detection (i.e., labeled specific binding agents and/or chromogenic compounds) and Giemsa stain. For example, kits for in situ hybridization procedures such as CISH include at least one target nucleic acid probe, at least one specific binding agent comprising an enzyme suitable for colorimetric detection, and at least one chromogen for use in colorimetric detection. In some embodiments, the kits further comprise other reagents for performing in situ hybridization such as paraffin pretreatment buffer, protease(s) and protease buffer, prehybridization buffer, hybridization buffer, wash buffer, counterstain(s), mounting medium, or combinations thereof. In some embodiments, the kits for ISH and chromosome banding include at least one target nucleic acid probe, reagents for ISH detection (i.e., labeled specific binding agents and/or chromogenic compounds), Giemsa stain, one or more cross-linking agents, paraffin pretreatment buffer, protease(s) and protease buffer, prehybridization buffer, hybridization buffer, wash buffer, counterstain(s), mounting medium, or combinations thereof.

The kit can optionally further include control slides for assessing hybridization and signal of the probe.

Likewise, the present invention provides automated systems for ISH and chromosome banding. In some preferred embodiments, the Ventana BenchMark XT™ instrument is adapted to include reservoirs and dispensers for Giemsa staining solutions as described above.

### EXAMPLES

### Example 1

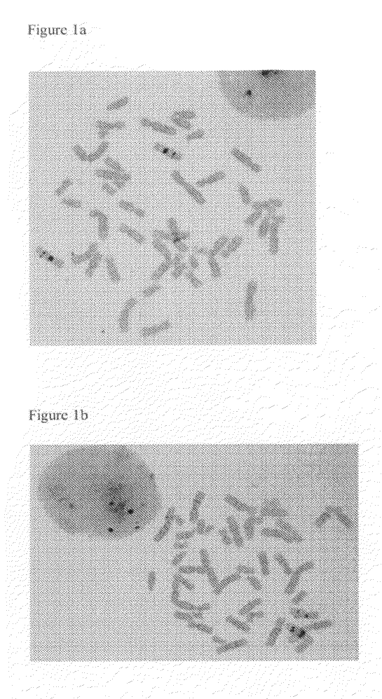

Metaphase chromosomes (CGH Metaphase Target Slides, Abbott Molecular, cat#30-806010) are UV cross-linked a in Stratalinker 2400 (Stratagene Model # C00518) at energy level of 200 mJ. 1% trypsin (Sigma cat#T1426) is added to the slides and the slides are incubated at room temperature for 5 s. The slides are rinsed with 1×PBS. The slides are placed on a Ventana BenchMark XT instrument for ISH staining. After the ISH staining is completed, the slides are rinsed with dawn detergent and deionized water. The slides are stained with 4% Giemsa (Gibco, cat#10092-03) diluted in Gurr buffer (Gibco, cat#10582-013) at room temperature for 5 min. The slides are rinsed with Dawn™ detergent deionized water. The slides are analyzed with a light microscope. FIGS. 1a and 1b are light micrographs of a sample that has been ISH-stained with a Met probe (black) and Chromosome 7 centromere probe (red) and banded.

### Example 2

Metaphase chromosomes (CGH Metaphase Target Slides, Abbott Molecular, cat#30-806010) are UV cross-linked a in Stratalinker 2400 (Stratagene Model # C00518) at energy level of 200 mJ. The slides are placed on a Ventana BenchMark XT instrument. 0.01% Trypsin is applied and the slides are incubated for 12 min. Following trypsinization, ISH is performed on the instrument. Giemsa (Ventana cat#860-006) is applied via the instrument and the slides are incubated at 37 C for 8 min. The slides are rinsed with Dawn™ detergent deionized water. The slides are analyzed with a light microscope.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method for in situ analysis of a sample, said method comprising:
contacting said sample with at least a first probe specific for a first target nucleic acid in said chromosomes under conditions such that said first probe hybridizes to said first target nucleic acid,
contacting said sample with in situ hybridization assay reagents,
banding said chromosomes to provide banded chromosomes, and
simultaneously analyzing said banded chromosomes for banding and hybridization of said first probe specific for said first target nucleic acid, wherein the presence of said first probe on said chromosome is indicated by said in situ hybridization assay reagents.

2. The method of claim 1, wherein said banding is performed by Giemsa staining said chromosomes.

3. The method of claim 1, wherein said first probe specific for said first target nucleic acid is conjugated to an enzyme that reacts with a colorimetric substrate and said in situ hybridization assay reagents comprise said colorimetric substrate.

4. (canceled)

5. The method of claim 3, wherein said enzyme that reacts with a colorimetric substrate is selected from the group consisting of horseradish peroxidase, alkaline phosphatase, acid phosphatase, glucose oxidase, β-galactosidase, β-glucuronidase and β-lactamase.

6. The method of claim 3, wherein said colorimetric substrate is selected from the group consisting of diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-3-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

7. The method of claim 1, wherein said first probe specific for said first target nucleic acid is conjugated to a hapten, and said in situ hybridization assay reagents comprise a specific binding reagent that binds to said hapten, said specific binding reagent comprising a signal generating moiety.

8. The method of claim 7, wherein said hapten is selected from the group consisting of biotin, 2,4-Dintropheyl (DNP), Fluorescein deratives, Digoxygenin (DIG), 5-Nitro-3-pyrozolecarbamide (nitropyrazole, NP), 4,5,-Dimethoxy-2-nitrocinnamide (nitrocinnamide, NCA), 2-(3,4-Dimethoxyphenyl)-quinoline-4-carbamide (phenylquinolone, DPQ), 2,1,3-Benzoxadiazole-5-carbamide (benzofurazan, BF), 3-Hydroxy-2-quinoxalinecarbamide (hydroxyquinoxaline, HQ), 4-(Dimethylamino)azobenzene-4′-sulfonamide (DABSYL), Rotenone isoxazoline (Rot), (E)-2-(2-(2-oxo-2,3-dihydro-1H-benzo[b][1,4]diazepin-4-yl)phenozy)acetamide (benzodiazepine, BD), 7-(diethylamino)-2-oxo-2H-chromene-3-carboxylic acid (coumarin 343, CDO), 2-Acetamido-4-methyl-5-thiazolesulfonamide (thiazolesulfonamide, TS), and p-Mehtoxyphenylpyrazopodophyllamide (Podo).

9. The method of claim 7, wherein said specific binding agent is conjugated to a signal generating moiety comprising an enzyme selected from the group consisting of horseradish peroxidase, alkaline phosphatase, acid phosphatase, glucose oxidase, β-galactosidase, β-glucuronidase and β-lactamase.

10. The method of claim 1, wherein said chromosomes are immobilized prior to said hybridization.

11. The method of claim 10, wherein said chromosomes are immobilized by cross-linking comprising exposure to ultraviolet radiation.

12. The method of claim 10, wherein said chromosomes are immobilized by cross-linking comprising exposure to a chemical cross-linking agent.

13. The method of claim 12, wherein said chemical cross-linking agent is selected from the group consisting of formaldehyde, glutaraldehyde, dimethyl suberimidate, dimethyl adipimidate, and N-hydroxysuccinimide esters.

14. The method of claim 1, further comprising enzymatically treating said sample prior to said hybridization step.

15. The method of claim 14, wherein said enzymatically treating said sample comprises treating with trypsin.

16. The method of claim 1, wherein said analyzing comprises viewing said sample with a light microscope.

17. The method of claim 1, wherein said analyzing comprises computer imaging said sample with a light microscope.

18. The method of claim 1, wherein said sample comprises cells fixed on a substrate.

19. The method of claim 17, wherein said cells are cells in a tissue section.

20. The method of claim 1, further comprising contacting said sample with a second probe specific for a second target nucleic acid in said chromosomes under conditions such that said second probe hybridizes to said second target nucleic acid and detecting said second probe.

21. (canceled)

22. An automated system for in situ analysis of a sample comprising chromosomes, said system comprising:
substrates compatible with fixation of said sample;
one or more probes specific for one or more target nucleic acids in said chromosomes;
colorimetric assay reagents for detection of said probes; and
banding reagents for banding said chromosomes.

23. A kit for in situ analysis of a sample comprising chromosomes, said system comprising:
one or more probes specific for one or more target nucleic acids in said chromosomes;
colorimetric assay reagents for detection of said probes; and
banding reagents for banding said chromosomes.
